(19) United States
(12) Patent Application Publication
(10) Pub. No.: US 2016/0250142 A1
(43) Pub. Date: Sep. 1, 2016

(54) INHALED AEROSOLIZED IMMUNO-CHEMOTHERAPY FOR THE TREATEMENT OF MDR TB

(71) Applicant: INSPIRX LLC, New Brunswick, NJ (US)

(72) Inventors: Rohinton D. TODDYWALA, Princeton, NJ (US); Sanjay B. BHARDWAJ, Cary, NC (US)

(73) Assignee: INSPIRX LLC, New Brunswick, NJ (US)

(21) Appl. No.: 15/033,190

(22) PCT Filed: Oct. 30, 2014
(86) PCT No.: PCT/US14/63082

§ 371 Date: Apr. 29, 2016

Related U.S. Application Data
(60) Provisional application No. 61/897,815, filed on Oct. 30, 2013.

(51) Int. Cl. A61K 9/00; A61K 31/5383; A61K 31/7036; A61K 47/20; A61K 47/26; A61K 47/24; A61K 47/12; A61K 38/21; A61K 31/4164

(52) U.S. Cl. CPC A61K 9/007; A61K 38/217; A61K 31/5383; A61K 31/7036; A61K 31/4164; A61K 47/26; A61K 47/24; A61K 47/12; A61K 47/20

(57) ABSTRACT

The present disclosure relates to the treatment of tuberculosis and/or multi-drug resistant tuberculosis by inhalable pharmaceutical compositions that include an interferon and at least one therapeutic agent selected from the group of fluoroquinolone, aminoglycoside and nitroimidazole. The present disclosure also relates to an inhalable pharmaceutical composition that includes an interferon and at least one therapeutic agent selected from the group of fluoroquinolone, aminoglycoside and nitroimidazole.

### PRIORITY

This application claims priority to U.S. Provisional Patent Application Ser. No. 61/897,815, filed Oct. 30, 2013; the contents of which are incorporated herein by reference.

### BACKGROUND OF THE DISCLOSURE

This disclosure relates to inhaled immuno-chemotherapy for the treatment of lung infections, including tuberculosis (“TB”), multi-drug resistant tuberculosis (“MDR TB”), mycobacterium avium complex (“MAC”), non-tuberculosis mycobacterial (“NTM”) pulmonary infections, rapid grower mycobacterium (“RGM”) (e.g. M. chelonae, M. abscessus, M. fortuitum), M. kansasii, and nosocomial infections, such as ventilator-assisted pneumonia.

In 1993, the World Health Organization (WHO) moved to classify TB as a global health emergency. Nearly two decades later, despite the progress made against stated millennial development goals, the statistics associated with the disease remain stunning: globally, almost 2 billion people (constituting nearly a third of the world's population) are infected with the latent form of the disease (LTBI—Latent TB infection), and about 10 percent of that incident population is expected to go on to develop the active infection.

While the reasons underlying emerging Mycobacterium tuberculosis (Mtb) drug resistance are complex, rational drug regimen selection and optimal antibiotic drug concentrations achieved at the site of infection (lung tissue) would facilitate early bactericidal efficacy and reduce the chances of survival, mutation and emergence of drug resistant Mtb strains. That being said, many of the “mainstay” drugs of escalating MDR TB treatment protocols (such as the injectable aminoglycoside antibiotics and orally administered fluoroquinolones) often exhibit poor penetration and suboptimal concentrations at the targeted lung tissue level due to their distribution pharmacokinetics. Furthermore, significant toxicities associated with systemic plasma exposure levels at higher doses preclude simple dose escalation strategies to achieve satisfactory bactericidal lung concentrations.

The WHO characterizes the global disease burden as enormous with 8.7 million new TB cases diagnosed in 2011 alone. The emergence of MDR TB as well as potentially totally-drug resistant TB suggests that TB could rapidly escalate to an existential threat to mankind.

Treatment of TB and MDR TB is often ineffective because patients express impaired immunity in the face of existing drug protocols that offer limited bioavailability at the site of infection and high oral toxicity profiles.

One potential method to alleviate poor penetration to the lungs and toxicity issues associated with treatment of tuberculosis is to deliver the pharmaceutical composition through an inhalation method.

Thus, there is a need for, inter alia, an effective inhaled MDR TB treatment that achieves high drug potency in the infected pulmonary tissue, and that are potentially less toxic due to lower circulating drug levels. To achieve the above, there is a need for a change in the route of administration from oral or parenteral to inhaled delivery (directly targeting the lungs) that can circumvent the associated pharmacokinetic, pharmacodynamic and toxicity constraints, effectively “repurposing” existing therapies, while significantly enhancing their safety and efficacy.

### SUMMARY OF THE DISCLOSURE

A method of treating tuberculosis, comprising administering, by inhalation, to a patient in need thereof a pharmaceutically acceptable amount of an interferon and at least one other therapeutic agent selected from the group of fluoroquinolones, aminoglycosides and nitroimidazoles; wherein the composition may be administered in combination or sequentially.

An inhalable pharmaceutical composition comprising an interferon and at least one other therapeutic agent selected from the group of fluoroquinolones, aminoglycosides and nitroimidazoles; wherein the composition has a pH of about 2 to about 8 and a tonicity of about 200 to about 800 mOsm.

An inhalable pharmaceutical composition comprising at least one therapeutic agent selected from the group of fluoroquinolones, aminoglycosides and nitroimidazoles; wherein the composition has a pH of about 2 to about 8 and a tonicity of about 200 to about 800 mOsm.

It is an object of the present invention to provide an inhaled delivery of a therapeutic formulation that is useful for the treatment of TB, especially MDR TB, MAC, NTM pulmonary infections, RGM, M. kansasii, and ventilator assisted pneumonia.

These, and other objectives as will be apparent to those of ordinary skill in the art, have been achieved by the administration of therapeutic agents, in aerosolized form.

The therapeutic agents include an immunomodulator with and chemotherapeutic agents that are active against TB and well as other lung infections. The therapeutic agents can be administered alone, sequentially or in combination with one another.

One sequential administration or combination includes interferon-gamma 1b, amikacin, levofloxacin and metronidazole.

With aerosolized delivery, localized drug concentrations delivered to the lung are considerably higher than that achievable by systemic administration and, in TB patients, the aerosol route has been proven to be more effective by achieving higher lung concentrations of drug compared to oral or injected delivery at the same or lower dose. Furthermore, in patients with active pulmonary tuberculosis, inhaled IFN-γ 1b has been shown to induce intracellular signaling of IFN-γ 1b specific transcription factors and to improve clinical response to anti-tuberculosis therapy.

### BRIEF DESCRIPTION OF THE DRAWINGS

FIG. 1. Graphical representation of the NGI impactor test for Metronidazole respiratory solution (8 mg/mL).

FIG. 2. Graphical representation of the NGI impactor test for Amikacin respiratory solution (200 mg/mL).

FIG. 3. Graphical representation of the NGI impactor test for Levofloxacin respiratory solution (250 mg/mL).

### DETAILED DESCRIPTION OF THE DISCLOSURE

The terms “compound(s)”, “pharmaceutical”, or “drug” according to the present disclosure include their tautomers, stereoisomers and mixtures thereof and the salts thereof, in particular the pharmaceutically acceptable salts thereof, and the solvates and hydrates of such compounds, including the solvates and hydrates of such tautomers, stereoisomers and salts thereof.

The terms “treat,” “treatment,” and “treating” embraces therapeutic, i.e. curative and/or palliative, treatment. Thus the terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The term “preventative” includes prophylactic treatment. The term “preventative” comprises treatment of patients that have not already developed a condition or at risk to develop a condition, thus reducing said risk.

When this disclosure refers to patients requiring treatment, it relates primarily to treatment in mammals, in particular humans.

By “therapeutically effective amount” or “pharmaceutically effective amount” is meant a compound or compounds, as disclosed for this invention, which has a therapeutic effect. The doses of compounds of the present disclosure which are useful in treatment are therapeutically effective amounts. Thus, as used herein, a therapeutically effective amount means those amounts of compounds which produce the desired therapeutic effect as judged by clinical trial results and/or model animal infection studies. In particular embodiments, the compounds are administered in a pre-determined dose, and thus a therapeutically effective amount would be an amount of the dose administered. This amount and the amount of the compound can be routinely determined by one of skill in the art, and will vary, depending on several factors, such as the particular microbial strain involved. This amount can further depend upon the patient's height, weight, sex, age and medical history. For prophylactic treatments, a therapeutically effective amount is that amount which would be effective to prevent a microbial infection.

A “therapeutic effect” relieves, to some extent, one or more of the symptoms of the infection, and includes, to some extent, curing an infection. “Curing” means that the symptoms of active infection are eliminated, including the total or substantial elimination of excessive members of viable microbes of those involved in the infection to a point at or below the threshold of detection by traditional measurements. However, certain long-term or permanent effects of the acute or chronic infection may exist even after a cure is obtained (such as extensive tissue damage). As used herein, a “therapeutic effect” is defined as a statistically significant reduction in bacterial load in a host, emergence of resistance, pulmonary function, or improvement in infection symptoms or functional status as measured by human clinical results or animal studies.

The terms “mediated” or “mediating” or “mediate”, as used herein, unless otherwise indicated, refers to the (i) treatment, including prevention of the particular disease or condition, (ii) attenuation, amelioration, or elimination of one or more symptoms of the particular disease or condition, or (iii) prevention or delay of the onset of one or more symptoms of the particular disease or condition described herein.

Unless specifically indicated, throughout the specification and the appended claims, a given chemical formula or name shall encompass tautomers and all stereo, optical and geometrical isomers (e.g. enantiomers, diastereomers, E/Z isomers etc. . . .) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobioic acid, tartaric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsufuric acid, naphthalene-1,5-disulfonic acid, naphthalene-2-sulfonic acid, and saccharin.

The term “administration” or “administering” refers to a method of giving a dosage of a pharmaceutical composition to a mammal, for example, by inhalation. The method of administration can vary depending on various factors, e.g., the components of the pharmaceutical composition, the site of the potential or actual bacterial infection, e.g. the lungs, the microbe involved, and the severity of an actual microbial infection.

A “carrier” or “excipient” is a compound or material used to facilitate administration of the compound, for example, to increase the solubility of the compound. Co-solvents include, e.g., water, ethanol, glycerin, propylene glycol and PEG 1000. Surfactants/lubricants include, e.g., sorbitan trioleate, soya lecithin, lecithin, oleic acid, magnesium stearate and sodium lauryl sulfate. Carrier particles include, e.g., lactose, mannitol and dextrose. Preservatives/antioxidants include, e.g., methylparaben, propylparaben, chlorobutanol, benzalkonium chloride, cetylpyridinium chloride, thymol, ascorbic acid, sodium bisulfate, sodium metabisulfite, sodium bisulfate and EDTA. Buffers/tonicity agents include, e.g., NaOH, tromethamine, ammonia, HCl, H₂SO₄, HNO₂, citric acid, CaCl₂ and CaCO₃. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, incorporated by reference herein in its entirety. Carriers and excipients that are deemed acceptable for pharmaceutical compounding of inhaled formulations form a subset of materials listed in the US National Formulary and under the FDA Database for Excipients acceptable for use in inhalation formulations.

The term “microbial infection” refers to the undesired proliferation or presence of invasion of pathogenic microbes in a host organism. This includes the excessive growth of microbes that are normally present in or on the body of a mammal or other organism, e.g. in the lungs. More generally, a microbial infection can be any situation in which the presence of a microbial population(s) is damaging to a host mammal. Thus, a microbial infection exists when excessive numbers of a microbial population are present in or on a mammal's body, or when the effects of the presence of a microbial population(s) is damaging the cells or other tissue of a mammal.

The term “chemotherapeutic agent” refers to a compound that is selectively toxic to and can be used to treat a disease, such as a virus, bacterium or other microorganism.

The term “sequentially” refers to the administration of more than one therapeutic agent at separate times. The therapeutic agents can be administered in any order. Unless the drugs are formulated together, they are considered to be administered sequentially. In one embodiment, two or more therapeutic agents are considered to be administered sequentially if they are administered within 24 hours of each other. In another embodiment, two or more therapeutic agents can be administered in less than a 24 hour period. In another embodiment, two or more therapeutic agents can be administered in less than a 12 hour period. In another embodiment, two or more therapeutic agents can be administered in less than a 6 hour period. In another embodiment, two or more therapeutic agents can be administered in less than a 3 hour period. In one embodiment, the therapeutic agents are administered immediately, one right after another. In another embodiment, the therapeutic agents are administered with an amount of time of about 5 minutes, about 10 minutes, about 15 minutes, about 20 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 6 hours, about 7 hours, about 8 hours, about 9 hours, about 10 hours, about 11 hours, about 12 hours, about 15 hours, about 18 hours, about 21 hours or about 24 hours in-between the administration of each therapeutic agent. For example, in one embodiment, separate formulations of interferon, amikacin, levofloxacin and metronidazole can all be administered within a 24 hour period, and they are considered to be administered sequentially.

The present disclosure relates to an aerosolized pharmaceutical combination of at least one immunomodulator with at least one chemotherapeutic agent that is active against TB. An immunomodulator is an active agent that is capable of treating a disease by inducing, enhancing or suppressing an immune response.

Immunomodulators are known in the art. Non-limiting examples of immunomodulators include interleukins, such as IL-2, IL-7 and IL-12; cytokines, such as interferon (“IFN”), IFN-α, IFN-β, IFN-ε, IFN-κ, IFN-ω, IFN-γ, and IFN-γ 1b; chemokines, such as CCL3, CCL26 and CXCL7; as well as cytosine phosphate-guanosine, oligodeoxynucleotides and glucans. In one embodiment, the immunomodulator is IFN-γ. In another embodiment, the immunomodulator is IFN-γ 1b.

Non-limiting examples of chemotherapeutic agents that are active against TB include aminoglycoside antibiotics, such as kanamycin A, amikacin, tobramycin, dibekacin, gentamicin, sisomicin, netilmicin, neomycin B, neomycin C, paromomycin and streptomycin; fluroquinolones, such as moxifloxacin, levofloxacin, sparfloxacin, nalidixic acid, ciprofloxacin, cinoxacin, oxolinic acid, piromidic acid, pipemidic acid, rosoxacin, enoxacin, fleroxacin, lomefloxacin, nadifloxacin, norfloxacin, ofloxacin, perfloxacin, rufloxacin, balofloxacin, grepafloxacin, pazufloxacin, temafloxacin, tosufloxacin, clinafloxacin, gatlifloxacin, sitafloxacin, prulifloxacin, delafloxacin, JNJ-Q2, nemofloxacin, danofloxacin, difloxacin, enrofloxacin, ibafloxacin, marbofloxacin, orbifloxacin, sarafloxacin and trovafloxacin; and nitroimidazole antibiotics, such as metronidazole, tinidazole and nimorazole. In one embodiment, the aminoglycoside antibiotic is amikacin. In one embodiment, the fluoroquinolone is selected from levofloxacin and moxifloxacin. In one embodiment, the nitroimidazole antibiotic is metronidazole.

In one embodiment, the pharmaceutical treatment includes administering, either sequentially or in combination, an immunomodulator and one chemotherapeutic agent. In another embodiment, the pharmaceutical treatment includes an immunomodulator and two chemotherapeutic agents. In yet another embodiment, the pharmaceutical treatment includes an immunomodulator and three chemotherapeutic agents. In yet another embodiment, the pharmaceutical treatment includes an immunomodulator and four or more chemotherapeutic agents. In one embodiment, the immunomodulator is IFN. In one embodiment the chemotherapeutic agent can be amikacin, levofloxacin or metronidzole.

In one embodiment, the pharmaceutical treatment includes administering, either alone, sequentially or in combination, one or more chemotherapeutic agents. In another embodiment, the pharmaceutical treatment includes administering, either alone, sequentially or in combination, two or more chemotherapeutic agents. In another embodiment, the pharmaceutical treatment includes administering, either alone, sequentially or in combination, three or more chemotherapeutic agents. In another embodiment, the pharmaceutical treatment includes administering, either alone, sequentially or in combination, four or more chemotherapeutic agents. In one embodiment the chemotherapeutic agent can be amikacin, levofloxacin or metronidzole.

In one embodiment, the pharmaceutical treatment includes an immunomodulator, e.g., IFN and an aminoglycoside, e.g., amikacin. In another embodiment, the pharmaceutical treatment includes an immunomodulator, e.g., IFN. In another embodiment, the pharmaceutical treatment includes a fluoroquinolone, e.g., levofloxacin. In another embodiment, the pharmaceutical treatment includes an aminoglycoside, e.g., amikacin. In another embodiment, the pharmaceutical treatment includes a nitroimidazole, e.g., metronidazole. In another embodiment, the pharmaceutical treatment includes an aminoglycoside, e.g., amikacin and a fluoroquinolone, e.g., levofloxacin. In another embodiment, the pharmaceutical treatment includes a nitroimidazole, e.g., metronidazole and a fluoroquinolone, e.g., levofloxacin. In another embodiment, the pharmaceutical treatment includes an aminoglycoside, e.g., amikacin and a nitroimidazole, e.g., metronidazole. In another embodiment, the pharmaceutical treatment includes an aminoglycoside, e.g., amikacin, a nitroimidazole, e.g., metronidazole and a fluoroquinolone, e.g., levofloxacin. In another embodiment, the pharmaceutical treatment includes an immunomodulator, e.g., IFN and a fluoroquinolone, e.g., levofloxacin. In another embodiment, the pharmaceutical treatment includes an immunomodulator, e.g., IFN and a nitroimidazole, e.g., metronidazole. In another embodiment, the pharmaceutical treatment includes an immunomodulator, e.g., IFN, amikacin and a fluoroquinolone, e.g., levofloxacin. In another embodiment, the pharmaceutical treatment includes an immunomodulator, e.g., IFN, amikacin and a nitroimidazole, e.g., metronidazole. In another embodiment, the pharmaceutical treatment includes an immunomodulator, e.g., IFN, a nitroimidazole, e.g., metronidazole and a fluoroquinolone, e.g., levofloxacin. In another embodiment, the pharmaceutical treatment includes an immunomodulator, e.g., IFN, an aminoglycoside, e.g., amikacin, a nitroimidazole, e.g., metronidazole and a fluoroquinolone, e.g., levofloxacin. In one embodiment, the compounds in the above combinations can be administered together at the same time or sequentially.

In one embodiment, the ratio of immunomodulator to aminoglycoside is about XX to about XX. In one embodiment, the ratio of immunomodulator to fluoroquinolone is about XX to about XX. In one embodiment, the ratio of immunomodulator to nitroimidazole is about XX to about XX. In one embodiment, the ratio of nitroimidazole to aminoglycoside is about XX to about XX. In one embodiment, the ratio of fluoroquinolone to aminoglycoside is about XX to about XX. In one embodiment, the ratio of fluoroquinolone to nitroimidazole is about XX to about XX.

It is contemplated by the present disclosure that part of the pharmaceutical treatment can be administered through inhalation while part of the combination can be administered through other means, e.g. orally or parenterally. However, in one embodiment, the components of the chemical formulation are intimately mixed together so that the immunomodulator, e.g. IFN, and at least one chemotherapeutic agent, e.g. one or more of amikacin, levofloxacin, metronidazole, and the like, are uniformly distributed throughout the formulation. In another embodiment, each therapeutic agent is separately formulated and administered either alone or sequentially with one or more therapeutic agent.

In one embodiment, the pharmaceutical treatment includes IFN-γ 1b, amikacin, levofloxacin and metronidazole.

Some embodiments include compositions comprised of an immunomodulator, such as IFN, and the like, with a fluoroquinolone, wherein the fluoroquinolone has an improved pulmonary availability, wherein an increased pulmonary AUC is indicative of the improved pulmonary availability of the fluoroquinolone relative to delivery of the fluoroquinolone through oral or parenteral administration. In some embodiments, the increase can be at least about 10% or more, about 20% or more, about 30% or more, about 40% or more, about 50% or more, about 75% or more, about 100% or more, about 150% or more, about 200% or more, about 250% or more, about 300% or more, and about 500% or more, wherein the increase can be relative to, for example, a composition delivered orally or parenteraly, and/or a composition delivered to the lung at a certain rate, and/or a certain respirable delivered dose. In some embodiments, methods are provided that include achieving an improved pulmonary availability of the fluoroquinolone indicated by a lung AUC of greater than about 400 mg/L, about 500 mg/L, about 600 mg/L, about 700 mg/L, about 800 mg/L, about 900 mg/L, about 1000 mg/L, about 1100 mg/L, about 1200 mg/L, about 1300 mg/L, about 1400 mg/L, about 1500 mg/L, about 1600 mg/L, about 1700 mg/L, about 1800 mg/L, about 1900 mg/L, about 2000 mg/L, about 2100 mg/L, about 2200 mg/L, about 2300 mg/L, about 2400 mg/L, about 2500 mg/L, about 2600 mg/L, about 2700 mg/L, about 2800 mg/L, about 2900 mg/L, about 3000 mg/L, about 3100 mg/L, about 3200 mg/L, about 3300 mg/L, about 3400 mg/L, about 3500 mg/L, about 3600 mg/L, about 3700 mg/L, about 3800 mg/L, about 3900 mg/L, about 4000 mg/L, about 4100 mg/L, about 4200 mg/L, about 4300 mg/L, about 4400 mg/L, and about 4500 mg/L. The increase can be measured for example, in bronchial fluid, homogenates of whole lung tissue, or in sputum.

Some embodiments include compositions comprised of an immunomodulator, such as IFN, and the like, with an aminoglycoside, wherein the aminoglycoside present has an improved pulmonary availability, wherein an increased pulmonary AUC is indicative of the improved pulmonary availability of the aminoglycoside relative to delivery of the aminoglycoside through oral or parenteral administration. In some embodiments, the increase can be at least about 10% or more, about 20% or more, about 30% or more, about 40% or more, about 50% or more, about 75% or more, about 100% or more, about 150% or more, about 200% or more, about 250% or more, about 300% or more, and about 500% or more, wherein the increase can be relative to, for example, a composition delivered orally or parenteraly, and/or a composition delivered to the lung at a certain rate, and/or a certain respirable delivered dose. In some embodiments, methods are provided that include achieving an improved pulmonary availability of the aminoglycoside indicated by a lung AUC of greater than about 400 mg/L, about 500 mg/L, about 600 mg/L, about 700 mg/L, about 800 mg/L, about 900 mg/L, about 1000 mg/L, about 1100 mg/L, about 1200 mg/L, about 1300 mg/L, about 1400 mg/L, about 1500 mg/L, about 1600 mg/L, about 1700 mg/L, about 1800 mg/L, about 1900 mg/L, about 2000 mg/L, about 2100 mg/L, about 2200 mg/L, about 2300 mg/L, about 2400 mg/L, about 2500 mg/L, about 2600 mg/L, about 2700 mg/L, about 2800 mg/L, about 2900 mg/L, about 3000 mg/L, about 3100 mg/L, about 3200 mg/L, about 3300 mg/L, about 3400 mg/L, about 3500 mg/L, about 3600 mg/L, about 3700 mg/L, about 3800 mg/L, about 3900 mg/L, about 4000 mg/L, about 4100 mg/L, about 4200 mg/L, about 4300 mg/L, about 4400 mg/L, and about 4500 mg/L. The increase can be measured for example, in bronchial fluid, homogenates of whole lung tissue, or in sputum.

Some embodiments include compositions comprised of an immunomodulator, such as IFN, and the like, with a nitroimidazole, wherein the nitorimidazole present has an improved pulmonary availability, wherein an increased pulmonary AUC is indicative of the improved pulmonary availability of the nitroimidazole relative to delivery of the nitroimidazole through oral or parenteral administration. In some embodiments, the increase can be at least about 10% or more, about 20% or more, about 30% or more, about 40% or more, about 50% or more, about 75% or more, about 100% or more, about 150% or more, about 200% or more, about 250% or more, about 300% or more, and about 500% or more, wherein the increase can be relative to, for example, a composition delivered orally or parenteraly, and/or a composition delivered to the lung at a certain rate, and/or a certain respirable delivered dose. In some embodiments, methods are provided that include achieving an improved pulmonary availability of the nitroimidazole indicated by a lung AUC of greater than about 400 mg/L, about 500 mg/L, about 600 mg/L, about 700 mg/L, about 800 mg/L, about 900 mg/L, about 1000 mg/L, about 1100 mg/L, about 1200 mg/L, about 1300 mg/L, about 1400 mg/L, about 1500 mg/L, about 1600 mg/L, about 1700 mg/L, about 1800 mg/L, about 1900 mg/L, about 2000 mg/L, about 2100 mg/L, about 2200 mg/L, about 2300 mg/L, about 2400 mg/L, about 2500 mg/L, about 2600 mg/L, about 2700 mg/L, about 2800 mg/L, about 2900 mg/L, about 3000 mg/L, about 3100 mg/L, about 3200 mg/L, about 3300 mg/L, about 3400 mg/L, about 3500 mg/L, about 3600 mg/L, about 3700 mg/L, about 3800 mg/L, about 3900 mg/L, about 4000 mg/L, about 4100 mg/L, about 4200 mg/L, about 4300 mg/L, about 4400 mg/L, and about 4500 mg/L. The increase can be measured for example, in bronchial fluid, homogenates of whole lung tissue, or in sputum.

For pulmonary administration, the upper airways are avoided in favor of the middle and lower airways. Pulmonary drug delivery can be accomplished by inhalation of an aerosol through the mouth and throat. Particles having a mass median aerodynamic diameter (MMAD) of greater than about 5 microns generally do not reach the lung; instead, they tend to impact the back of the throat and are swallowed and possibly orally absorbed. Particles having diameters of about 2 to about 5 microns are small enough to reach the upper- to mid-pulmonary region (conducting airways), but are too large to reach the alveoli. Smaller particles, i.e., about 0.5 to about 2 microns, are capable of reaching the alveolar region. Particles having diameters smaller than about 0.5 microns can also be deposited in the alveolar region by sedimentation, although very small particles may be exhaled.

In one embodiment, a nebulizer is selected on the basis of allowing the formation of an aerosol of the pharmaceutical combination disclosed herein having an MMAD predominantly between about 0.5 to about 5 microns. In one embodiment, the delivered amount of the pharmaceutical combination provides a therapeutic effect for respiratory infections. The nebulizer can deliver an aerosol comprising a mass median aerodynamic diameter from about 0.5 microns to about 5 microns, a mass median aerodynamic diameter from about 1.0 microns to about 3.0 microns, or a mass median aerodynamic diameter from about 1.5 microns to about 2.5 microns. In some embodiments, the MMAD can be about 0.5 microns, about 1.0 microns, about 1.5 microns, about 2.0 microns, about 2.5 microns, about 3.0 microns, about 3.5 microns, about 4.0 microns, about 4.5 microns or about 5.0 microns. In one embodiment, the MMAD ranges from about 2.5 to about 5.0 microns. In another embodiment, the MMAD ranges from about 3.0 to about 4.5 microns. In some embodiments, the nebulizer can be a breath actuated nebulizer (BAN). In some embodiments, the aerosol can be produced using a vibrating mesh nebulizer. An example of a vibrating mesh nebulizer includes the PARI E-FLOW® nebulizer or a nebulizer using PARI eFlow technology. More commercial examples of nebulizers that can be used with the formulations described herein include Respirgard II®, Aeroneb®, Aeroneb Pro®, Aeroneb Go®, AERx®, AERx Essence®, Porta-Neb®, Freeway Freedom®, Sidestream®, Ventstream®, I-neb®, PARI LC-Plus®, and PARI LC-Start®. In one embodiment, the nebulizer is a breath actuated nebulizer.

The amount of fluoroquinolone that can be administered to the lungs with an aerosol dose, such as a respirable drug dose (RDD), can include about 0.01 mcg, about 0.02 mcg, about 0.03 mcg, about 0.04 mcg, about 0.05 mcg, about 0.1 mcg, about 0.2 mcg, about 0.5 mcg, about 1 mcg, about 2 mcg, about 5 mcg, about 10 mcg, about 20 mcg, about 30 mcg, about 40 mcg, about 50 mcg, about 60 mcg, about 70 mcg, about 80 mcg, about 90 mcg, about 100 mcg, about 150 mcg, about 200 mcg, about 300 mcg, about 400 mcg, about 500 mcg, about 600 mcg, about 700 mcg, about 800 mcg, about 900 mcg, about 1 mg, about 2 mg, about 5 mg, about 10 mg, about 15 mg, 20 mg, about 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg. In some embodiments, the amount of fluoroquinolone that can be administered to the lungs with an aerosol dose, such as a respirable drug dose (RDD), can include about 0.01 mcg, about 0.02 mcg, about 0.03 mcg, about 0.04 mcg, about 0.05 mcg, about 0.1 mcg, about 0.2 mcg, about 0.5 mcg, about 1 mcg, about 2 mcg, about 5 mcg, about 10 mcg, about 20 mcg, about 30 mcg, about 40 mcg, about 50 mcg, about 60 mcg, about 70 mcg, about 80 mcg, about 90 mcg, about 100 mcg, about 150 mcg, about 200 mcg, about 300 mcg, about 400 mcg, about 500 mcg, about 600 mcg, about 700 mcg, about 800 mcg, about 900 mcg, about 1 mg, about 2 mg, about 5 mg, about 10 mg, about 15 mg, about 20 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg.

The amount of aminoglycoside that can be administered to the lungs with an aerosol dose, such as a respirable drug dose (RDD), can include about 1 mg, about 2 mg, about 5 mg, about 10 mg, about 15 mg, 20 mg, about 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg. In some embodiments, the amount of aminoglycoside that can be administered to the lungs with an aerosol dose, such as a respirable drug dose (RDD), can include about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg.

The amount of nitroimidazole that can be administered to the lungs with an aerosol dose, such as a respirable drug dose (RDD), can include about 1 mcg, about 2 mcg, about 5 mcg, about 10 mcg, about 20 mcg, about 30 mcg, about 40 mcg, about 50 mcg, about 60 mcg, about 70 mcg, about 80 mcg, about 90 mcg, about 100 mcg, about 150 mcg, about 200 mcg, about 300 mcg, about 400 mcg, about 500 mcg, about 600 mcg, about 700 mcg, about 800 mcg, about 900 mcg, about 1 mg, about 2 mg, about 5 mg, about 10 mg, about 15 mg, 20 mg, about 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg. In some embodiments, the amount of nitroimidazole that can be administered to the lungs with an aerosol dose, such as a respirable drug dose (RDD), can include about 1 mcg, about 2 mcg, about 5 mcg, about 10 mcg, about 20 mcg, about 30 mcg, about 40 mcg, about 50 mcg, about 60 mcg, about 70 mcg, about 80 mcg, about 90 mcg, about 100 mcg, about 150 mcg, about 200 mcg, about 300 mcg, about 400 mcg, about 500 mcg, about 600 mcg, about 700 mcg, about 800 mcg, about 900 mcg, about 1 mg, about 2 mg, about 5 mg, about 10 mg, about 15 mg, about 20 mg, about 50 mg, about 100 mg, about 150 mg, about 200 mg, about 250 mg, about 300 mg, about 350 mg, about 400 mg, about 450 mg, or about 500 mg,.

The amount of interferon that can be administered to the lungs with an aerosol dose, such as a respirable drug dose (RDD), can include about 0.01 mcg, about 0.02 mcg, about 0.03 mcg, about 0.04 mcg, about 0.05 mcg, about 0.1 mcg, about 0.2 mcg, about 0.5 mcg, about 1 mcg, about 2 mcg, about 5 mcg, about 10 mcg, about 20 mcg, about 30 mcg, about 40 mcg, about 50 mcg, about 60 mcg, about 70 mcg, about 80 mcg, about 90 mcg, about 100 mcg, about 150 mcg, about 200 mcg, about 300 mcg, about 400 mcg, about 500 mcg, about 600 mcg, about 700 mcg, about 800 mcg, about 900 mcg, or about 1 mg. In some embodiments, the amount of interferon that can be administered to the lungs with an aerosol dose, such as a respirable drug dose (RDD), can include about 0.01 mcg, about 0.02 mcg, about 0.03 mcg, about 0.04 mcg, about 0.05 mcg, about 0.1 mcg, about 0.2 mcg, about 0.5 mcg, about 1 mcg, about 2 mcg, about 5 mcg, about 10 mcg, about 20 mcg, about 30 mcg, about 40 mcg, about 50 mcg, about 60 mcg, about 70 mcg, about 80 mcg, about 90 mcg, about 100 mcg, about 150 mcg, about 200 mcg, about 300 mcg, about 400 mcg, about 500 mcg, about 600 mcg, about 700 mcg, about 800 mcg, about 900 mcg, or about 1 mg.

The formulation can have a pH from about 1.0 to about 10.5, or from about 2.0 to about 8.0, or from about 1.5 to about 6.5, or from about 3.0 to about 7.0, or from about 5.0 to about 8.0, or from about 5.0 to about 7.0, or from about 5.0 to about 6.5, or from about 5.5 to about 6.5, or from about 6.0 to about 6.5. In some embodiments, the formulation can have a pH of about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 or 10.5.

The formulations, containing one or more therapeutic agent, can have a tonicity from about 50 to about1,000 mOsm, or from about 200 to about 800 mOsm, or from about 200 to about 600 mOsm. In some embodiments, the formulation can have a tonicity of about 50, about 100 mOsm, about 150 mOsm, about 200 mOsm, about 250 mOsm, about 300 mOsm, about 350 mOsm, about 400 mOsm, about 450 mOsm, about 500 mOsm, about 550 mOsm, about 600 mOsm, about 650 mOsm, about 700 mOsm, about 750 mOsm, about 800 mOsm, about 850 mOsm, about 900 mOsm, about 950 mOsm or about 1,000 mOsm.

The formulation can comprise a conventional pharmaceutical carrier, excipient or the like that are approved for inclusion in inhaled products per the US National Formulary and database of approved excipients maintained by USFDA and other regulatory agencies. Non-limiting examples of carriers and excipients include, e.g., water, ethanol, glycerin, propylene glycol, PEG 1000, sorbitan trioleate, soya lecithin, lecithin, oleic acid, magnesium stearate, sodium lauryl sulfate, lactose, mannitol, dextrose, methylparaben, propylparaben, chlorobutanol, benzalkonium chloride, cetylpyridinium chloride, thymol, ascorbic acid, sodium bisulfate, sodium metabisulfite, sodium bisulfate, EDTA, NaOH, tromethamine, ammonia, HCl, H₂SO₄, HNO₂, citric acid, CaCl₂ and CaCO₃.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution or suspension. Solutions to be aerosolized can be prepared in conventional forms, either as liquid solutions or suspensions, as emulsions, or in solid forms suitable for dissolution or suspension in liquid prior to aerosol production and inhalation. The percentage of active compound contained in such aerosol compositions is highly dependent on the specific nature thereof, as well as the activity of the compound and the needs of the subject. However, percentages of active ingredient(s) of about 0.01% to about 90% in solution are employable, and will be higher if the composition is a solid, which will be subsequently diluted to the above percentages. In some embodiments, the composition will comprise 1.0%-50.0% of the active agent(s) in solution. In some embodiments, the composition will comprise about 1.0, 2.0, 3.0, 4.0, 5.0, 10.0, 15.0, 20.0, 25.0, 30.0, 35.0, 40.0, 45.0, 50.0, 55.0, 60.0, 65.0, 70.0, 75.0, 80.0, 85.0 or 90.0% of the active agent(s) in solution.

The formulation may be administered at a therapeutically effective dosage, e.g., a dosage sufficient to provide treatment for the disease states previously described. The amount of active compound administered will, of course, be dependent on the subject and disease state being treated, the severity of the affliction, the manner and schedule of administration and the judgment of the prescribing physician.

Administration of the formulations disclosed herein or the pharmaceutically acceptable salts thereof can be via any of the accepted modes of administration for agents that serve similar utilities including, but not limited to, nebulized or aerosol inhalation. In one embodiment, the administration of the formulation is by breath actuated nebulization. The pharmaceutical combination can be delivered by inhalation by dry powder inhalers, such as Aerolizer, Diskus, Flexhaler, Handihaler, Neohaler, Pressair, Rotahaler, Tubuhaler and Twisthaler; metered-dose inhalers; and nebulizers, such as a breath-actuated wet nebulizer, soft mist inhaler, human powered nebulizer, vibrating mesh nebulizer, jet nebulizer and ultrasonic wave nebulizer. Aerosols can be delivered using metered dose inhalers (pMDI's), nebulizers or dry powder inhalers (DPI's). pMDI's and DPI's can be expensive and offer significant complications when used with combination products. Wet nebulization can offer a simple, cost effective way of delivering aerosols especially when there are multiple drugs involved. Current Jet Nebulizers although cost effective, do not provide reproducible doses of medication and lead to significant residual volumes (i.e., wastage of drug). Further, Jet Nebulizers operate continuously and therefore could be unsafe for the clinician/caregiver who may also breathe in the aerosol. The medicine is formulated as a suspension or solution of a drug substance in a suitable propellant such as a halogenated hydrocarbon. There are two major designs of dry powder inhalers. One design is the metering device in which a reservoir for the drug is placed within the device and the patient adds a dose of the drug into the inhalation chamber. The second is a factory-metered device in which each individual dose has been manufactured in a separate container.

In some embodiments, solid drug nanoparticles are provided for use in generating dry aerosols or for generating nanoparticles in liquid suspension. Powders comprising nanoparticulate drug can be made by spray-drying aqueous dispersions of a nanoparticulate drug and a surface modifier to form a dry powder which consists of aggregated drug nanoparticles. In one embodiment, the aggregates can have a size of about 0.5 to about 2.5 microns which is suitable for deep lung delivery. In another embodiment, the aggregates can have a size of about 2.5 to about 5.0 microns. The aggregate particle size can be increased to target alternative delivery sites, such as the upper bronchial region or nasal mucosa by increasing the concentration of drug in the spray-dried dispersion or by increasing the droplet size generated by the spray dryer. In some embodiments, pharmaceutical compounds disclosed herein may be formulated into liposome particles, which can then be aerosolized for inhaled delivery. Lipids which are useful in the present invention can be any of a variety of lipids including both neutral lipids and charged lipids. Carrier systems having desirable properties can be prepared using appropriate combinations of lipids, targeting groups and circulation enhancers. Microspheres can be used for pulmonary delivery of pharmaceutical compounds by first adding an appropriate amount of drug compound to be solubilzed in water.

Unlike commercial jet nebulizers, the breath-actuated nebulizer is designed to create aerosol in response to the patient's inspiratory pattern. This patient on-demand therapy will mean less medication waste, higher drug delivery efficiency and safer clinician/caregiver working environments especially for high potency drugs. Clinician/caregiver-friendly improvements sustain aerosol output and enhance breath actuation while delivering a high respirable dose and making it possible to reduce treatment times per patient.

By non-limiting example, classes of taste-masking agents for the present formulations include the addition of flavorings, sweeteners, and other various coating strategies. By non-limiting examples these may be chosen from sugars such as sucrose, dextrose, and lactose), carboxylic acids, salts such as magnesium and calcium (non-specific or chelation-based fluoroquinolone taste masking), menthol, amino acids or amino acid derivatives such as arginine, lysine, and monosodioum glutamate, and synthetic flavor oils and flavoring aeromatics and/or natural oils, extracts from plants, leaves, flowers, fruits, etc. and combinations thereof. These may include cinnamon oils, oil of wintergreen, peppermint oils, clover oil, bay oil, anise oil, eucalyptus, vanilla, citrus oil such as lemon oil, orange oil, grape and grapefruit oil, fruit essences including apple, peach, pear, strawberry, raspberry, cherry, plum, pineapple, apricot, etc. Additional sweeteners include sucrose, dextrose, aspartame, acesulfame-K, sucrolose and saccharin, organic acids (by non-limiting example citric acid and aspartic acid). Such flavors may be present at about 0.05 to about 4 percent. Another approach to improve or mask the taste of unpleasant inhaled drugs is to decrease the drugs solubility, e.g. drugs must dissolve to interact with taste receptors. Hence, to deliver solid forms of the drug may avoid the taste response and acquire the desired improved taste affect. Non-limiting methods to decrease pharmaceutical compound solubility are described in this document, e.g. salt forms of the compound with xinafoic acid, oleic acid, stearic acid and pamoic acid. Additional co-precipitating agents include dihydropyridines and a polymer such as polyvinyl pyrrolidone. Moreover, taste-masking may be accomplished by creation of lipopilic vesicles. Additional coating or capping agents include dextrates (by non-limiting example cyclodextrins may include, 2-hydroxypropyl-beta-cyclodextrin, 2-hydroxypropyl-gamma-cyclodextrin, randomly methylated beta-cyclodextrin, dimethyl-alpha-cyclodextrin, dimethyl-beta-cyclodextrin, maltosyl-alpha-cyclodextrin, glucosyl-1-alpha-cyclodextrin, glucosyl-2-alpha-cyclodextrin, alpha-cyclodextrin, beta-cyclodextrin, gamma-cyclodextrin, and sulfobutylether-beta-cyclodextrin), modified celluloses such as ethyl cellulose, methyl cellulose, hydroxypropyl cellulose, hydroxyl propyl methyl cellulose, polyalkylene glycols, polyalkylene oxides, sugars and sugar alcohols, waxes, shellacs, acrylics and mixtures thereof. By non-limiting example, other methods to deliver non-dissolved forms of pharmaceutical compounds are to administer the drug alone or in simple, non-solubilty affecting formulation as a crystalline micronized, dry powder, spray-dried, and nanosuspension formulation. However, an alternative method is to include taste-modifying agents. These include taste-masking substance that is mixed with, coated onto or otherwise combined with the pharmaceutical compounds. However, this addition may also serve to improve the taste of another chosen drug product addition, e.g. a mucolytic agent. Non-limiting examples of such substances include acid phospholipids, lysophospholipid, tocopherol polyethyleneglycol succinate, and embonic acid (pamoate). Many of these agents can be used alone or in combination with pharmaceutical compounds for aerosol administration.

In one embodiment, the formulations, with one or more therapeutic agents, can be administered to the lungs in less than about 60 minutes, about 55 minutes, about 50 minutes, about 45 minutes, about 40 minutes, about 35 minutes, about 30 minutes, about 25 minutes, about 20 minutes, about 15 minutes, about 10 minutes, about 5 minutes, about 4 minutes, about 3 minutes, about 2 minutes, and about 1 minute.

Methods and compositions described herein can be used to treat pulmonary infections and disorders besides tuberculosis. Examples of other such disorders can include cystic fibrosis, pneumonia, and chronic obstructive pulmonary disease, including chronic bronchitis, and some asthmas. Some embodiments include treating an infection comprising one or more bacteria selected from the group consisting of Pseudomonas aeruginosa, Pseudomonas fluorescens, Pseudomonas acidovorans, Pseudomonas alcaligenes, Pseudomonas putida, Stenotrophomonas maltophilia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Helicobacter pylori, Campylobacter fetus, Campylobacter jejuni, Campylobacter coli, Borrelia burgdorferi, Vibrio cholera, Vibrio parahaemolyticus, Legionella pneumophila, Listeria monocytogenes, Neisseria gonorrhoeae, Neisseria meningitidis, Burkholderia cepacia, Francisella tularensis, Kingella, and Moraxella. In some embodiments, the lung infection is caused by a gram-negative anaerobic bacteria. In more embodiments, the lung infection comprises one or more of the bacteria selected from the group consisting of Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, and Bacteroides splanchnicus. In some embodiments, the lung infection is caused by a gram-positive bacteria. In some embodiments, the lung infection comprises one or more of the bacteria selected from the group consisting of Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, Enterococcus faecium, Staphylococcus aureus, Staphylococcus epidermidis, Staphylococcus saprophyticus, Staphylococcus intermedius, Staphylococcus hyicus, Staphylococcus haemolyticus, Staphylococcus hominis, and Staphylococcus saccharolyticus. In some embodiments, the lung infection is caused by gram-positive anaerobic bacteria. In some embodiments, the lung infection is caused by one or more bacteria selected from the group consisting of Clostridium difficile, Clostridium perfringens, Clostridium tetini, and Clostridium botulinum. In some embodiments, the lung infection is caused by an acid-fast bacteria. In some embodiments, the lung infection is caused by one or more bacteria selected from the group consisting of Mycobacterium tuberculosis, Mycobacterium avium, Mycobacterium intracellulare, and Mycobacterium leprae. In some embodiments, the lung infection is caused by atypical bacteria. In some embodiments, the lung infection is caused by one or more bacteria selected from the group consisting of Chlamydia pneumoniae and Mycoplasma pneumoniae.

Solutions containing the compounds for the disclosed pharmaceutical combination may be prepared together or separately and later combined.

In one embodiment, the amount of amikacin in the formulation is about 200 to about 250 mg/mL or total nebulization dose of about 600 to about 1250 mg per 3 or 5 mL nebule administered to the patient. In another embodiment, the pH of the amikacin solution is about 3 to about 4. In one embodiment, the amikacin solution has a tonicity of about 50 to about 1,000 mOsm. In another embodiment, the amikcacin solution has a tonicity of about 200 to about 500 mOsm.

In one embodiment, the amount of levofloxacin in the formulation is about 100 to about 250 mg/mL or total nebulization dose of about 300 to about 1250 mg per 3 or 5 mL nebule administered to the patient. In another embodiment, the pH of the levofloxacin solution is about 6 to about 7. In one embodiment, the levofloxacin solution has a tonicity of about 50 to about 500 mOsm. In another embodiment, the levofloxacin solution has a tonicity of about 100 to about 250 mOsm.

In one embodiment, the amount of metronidazole in the formulation is about 50 mg/mL or total nebulization dose of about 150 to about 250 mg per 3 or 5 mL nebule administered to the patient. In another embodiment, the pH of the metronidazole solution is about 1.5 to about 2.5. In one embodiment, the metronidazole solution has a tonicity of about 50 to about 1,000 mOsm. In another embodiment, the metronidazole solution has a tonicity of about 200 to about 400 mOsm.

In one embodiment, the amount of interferon in the formulation is about 0.01 to about 33 mcg/mL or total nebulization dose of about 0.03 to about 165 mcg per 3 or 5 mL nebule administered to the patient. In another embodiment, the pH of the interferon solution is about 1 to about 8. In one embodiment, the interferon solution has a tonicity of about 100 to about 1,000 mOsm. In another embodiment, the interferon solution has a tonicity of about 200 to about 800 mOsm.

In one embodiment, the formulation includes an amount of amikacin in the formulation of 200-250 mg/mL or total nebulization dose of 600-1250 mg per 3 or 5 mL nebule; an amount of levofloxacin in the formulation of 100-250 mg/mL or total nebulization dose of 300-1250 mg per 3 or 5 mL nebule; an amount of metronidazole in the formulation of 50 mg/mL or total nebulization dose of 150-250 mg per 3 or 5 mL nebule; an amount of interferon in the formulation of 0.01-33 mcg/mL or total nebulization dose of 0.03-165 mcg per 3 or 5 mL nebule; a pH of 1-8 and a tonicity of 200-800.

### EXAMPLES

### Example 1

HPLC methods were developed for Amikacin, Levofloxacin and Metronidazole. For Levofloxacin and Metronidazole a standard USP HPLC assay method was tested and found to be appropriate. Since the analytical method for Amikacin required derivatization a literature-based method was developed and used. Table 1 shows a summary of the HPLC methods used.

Table 2 summarizes different formulations that were made and the tested ranges.

TABLE 2

| | Amikacin | | Levofloxacin | | Metronidazole | |
|---|---|---|---|---|---|---|
| Nebulization | 200-250 mg/mL as a buffered | | 150-250 | mg/mL | 6-10 mg/mL as a buffered solution of pH | |
| Dose | solution | | | | 4.5 | |
| | 200-250 mL solution as an | | | | 50-60 mg/mL as a acidified solution | |
| | acidified solution | | | | | |
| pH | 3-4 | | 6-7 | | 4.6 | |
| | | | | | 1.5-2.5 | |
| Osmolarity | 200-500 | mOsm | 100-250 | mOsm | 20 | mOsm |
| | | | | | 200-400 | mOsm |
| Buffer Strength | N/A | | N/A | | 10 | mM |
| | | | | | 100 | mM |
| Nebulization | 3-5 | mL | 3-5 | mL | 3-5 | mL |
| Volume | | | | | | |

| | | | |
|---|---|---|---|
| Appearance | Clear colorless solution | Clear yellowish solution | Clear colorless solution |
| Particle Size | 200 mg/mL solution | 250 mg/mL Solution | 8 mg/mL solution |
| (@ 15 L/min) | MMAD - 3.3 microns | MMAD - 4.1 microns | MMAD - 3.9 |
| | GSD - 1.7 | GSD - 1.8 microns | GSD - 1.9 |

Table 3 shows a summary of formulations used for a pre-clinical study.

TABLE 3

| | Amikacin | | Levofloxacin | | Metronidazole | |
|---|---|---|---|---|---|---|
| Nebulization | 240 | mg/mL | 150 | mg/mL | 50 | mg/mL |
| Dose | | | | | | |
| Appearance | Clear colorless | | Clear yellowish | | Clear colorless | |
| | solution | | solution | | solution | |
| pH | 6.0 | | 4.0 | | 2.0 | |
| Osmolarity | 250 | mOsm | 150 | mOsm | 375 | mOsm |

Table 4 shows study results from formulations prior to and after the completion of a pre-clinical study.

TABLE 4

| Formulation | Properties | | | After completion | |
|---|---|---|---|---|---|
| Prepared | observed | Initial | | of study | |
| 50 mg/mL | Color, Odor, | Clear | | Clear | |
| Metronidazole | Appearance | | | | |
| in sulfuric | pH | 1.72 | | 1.70 | |
| acid matrix , | Osmolality | 397 | mOsm/kg | 399 | mOsm/kg |
| pH = 1.72 | Assay | 50.00 | mg/mL | 49.5 | mg/mL |
| 240 mg/mL | Color, Odor, | Clear | | Clear | |
| Amikacin | Appearance | | | | |
| Sulfate | pH | 4.10 | | 4.11 | |
| pH = 4.10 | Osmolality | 576 | mOsm/kg | 582 | mOsm/kg |
| | Assay | 243.655 | mg/mL | 271.0 | mg/mL |
| 150 mg/mL | Color, Odor, | Clear | | Clear | |
| Levofloxacin | Appearance | | | | |
| in sulfuric | pH | 6.13 | | 6.18 | |
| acid matrix, | Osmolality | 145 | mOsm/kg | 145 | mOsm/kg |
| pH = 6.13 | Assay | 155.489 | mg/mL | 148.8 | mg/mL |

Table 5 shows excipients used in formulating amikacin, levofloxacin, metronidazole and interferon gamma.

TABLE 5

| | Levofloxacin | Metronidazole | Interferon- |
|---|---|---|---|
| Amikacin Sulfate | Hemihydrate | Nebulization | gamma 1b |
| Nebulization Solution | Nebulization Solution | Solution | Nebulization Solution* |
| Sodium Citrate Dihydrate, USP | Sulfuric Acid, NF | Sulfuric Acid, NF | Mannitol, USP |
| Sulfuric Acid, NF | Sterile Filtered Water | Sterile Filtered Water | Sodium Succinate, |
| Sterile Filtered Water | | | Polysorbate 20, NF |
| | | | Sterile Filtered Water |

### Example 2

Formulations prepared for a pre-clinical study were observed at room temperature for a period of 10 weeks. The results are shown in Table 6. The formulations appeared to be stable based on a stability assessment, although the amikacin formulation decreased in terms of its assay. Some degradation is normal and expected for solution formulations of antibiotics.

TABLE 6

| Formulation | Properties | Initial | | 5-week | | 10-week | |
|---|---|---|---|---|---|---|---|
| Prepared | observed | 27 Jun. 2014 | | 5 Aug. 2014 | | 10 Sep. 2014 | |
| 50 mg/mL | Color, Odor, | Clear | | Clear | | Clear | |
| Metronidazole | Appearance | | | | | | |
| in sulfuric | pH | 1.97 | | 1.79 | | NM* | |
| acid matrix, | Osmolality | 369 | mOsm/kg | 379 | mOsm/kg | NM* | |
| pH = 1.97 | Assay | 50.00 | mg/mL | 50.3 | mg/mL | 50.1 | mg/mL |
| 240 mg/mL | Color, Odor, | Clear | | Slight color and | | Slight color and | |
| Amikacin | Appearance | | | start of precipitates | | start of precipitates | |
| Sulfate in 2.5% | pH | 4.08 | | 4.18 | | NM* | |
| Citrate buffer, | Osmolality | 577 | mOsm/kg | 574 | mOsm/kg | NM* | |
| pH = 4.08 | Assay | 243.855 | mg/mL | 195.0 | mg/mL | 227.0 | mg/mL |
| 150 mg/mL | Color, Odor, | Clear Deep Yellow | | Clear Deep Yellow | | Clear Deep Yellow | |
| Levofloxacin | Appearance | | | | | | |
| in sulfuric | pH | 5.96 | | 6.11 | | NM* | |
| acid matrix, | Osmolality | 142 | mOsm/kg | 143 | mOsm/kg | NM* | |
| pH = 5.96 | Assay | 155.489 | mg/mL | 153.4 | mg/mL | 150.2 | mg/mL |

NM* Not measured due to insufficient sample size

### Example 3

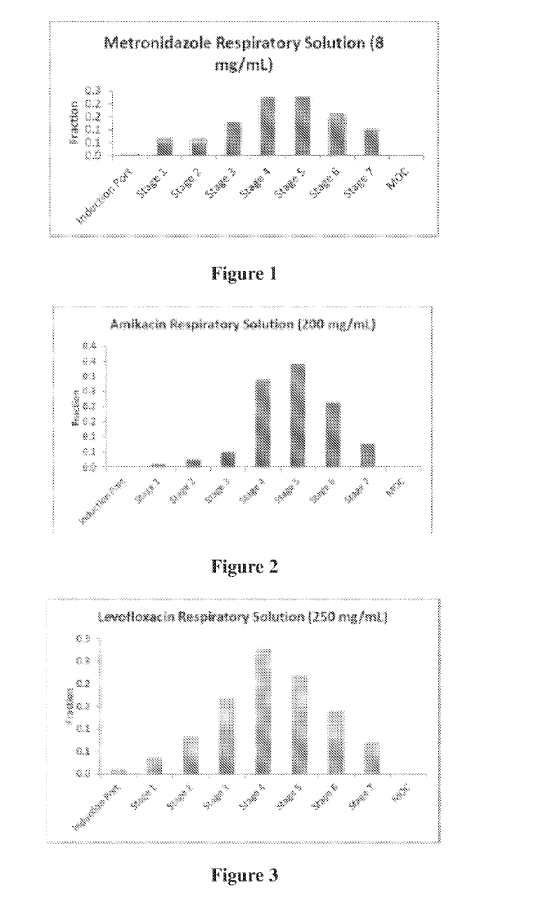

The nebulization drug formulations were nebulized and passed through an NGI impactor (Westech Corporation). These experiments show the fraction of drug in various stages of the impactor (FIGS. 1-3). It was shown that significant amounts of the drug compounds were obtained at the different stages, which corresponds to the respirable fraction that would be deposited in the lungs. The drug fractions were analyzed by HPLC. It was shown that metronidazole, amikacin and levofloxacin can be nebulized to produce droplets in the respirable range, but the profile is influenced by formulation characteristics, with solution viscosity and drug loading concentration playing an influencing role.

### Example 4

Pharmacokinetic (PK) testing was performed in mice to quantitatively assess drug distribution, efficacy and toxicity. The PK characteristics following lung deposition of the drug dose, distribution half life, chronological lung concentration versus required minimum inhibitory concentration (MIC) level for the tested drugs and the initial inhaled toxicity assessment of the drugs were all observed. Observations were taken at 1 and 8 hour time points within lung tissue as well as the plasma of individual animals.

A microsprayer delivery device was selected (instead of nebulized delivery) to minimize confounding variables. Its use eliminated experimental variables introduced by the use of nebulizers (aerosol profile, device variables, animal inspiration rate, errors in estimation of actual delivered dose, etc.).

In order to simulate drug delivery losses typically experienced in nebulized drug delivery, the actual dose delivered to the mice was reduced by 50% (via dilution of the nebulized solutions) to better simulate real-world drug delivery “dose attrition.” Typically, approximately half of the nominal dose loaded into a nebulizer device is lost due to a number of factors that contribute to cumulative drug payload losses during actual lung delivery. These include variables such as the volume of nebulization retained within the “dead space” of the nebulizer, throat deposition and additional confounding factors such as aerosolization and lung deposition losses caused by suboptimal aerosol particle size distribution.

The Provantis application software (Instem Life Sciences Systems, Ltd.; Staffordshire, United Kingdom) was used for the direct on-line capture of most in-life data. Environmental monitoring of the animal rooms (i.e., temperature/humidity and light/dark cycles) was performed using the Edstrom Watchdog system (Edstrom Industries, Inc.; Waterford, Wis.). The remainder of the data was collected manually.

24 female C57BL/6 mice designated for use on this study were selected from 28 mice obtained from Charles River Laboratories (Raleigh, N.C.). The mice were approximately 13 weeks of age when received at Southern Research Institute (Southern Research). The mice were housed under A/BSL-1 containment upon arrival and were observed for general health and acceptability for use in this study prior to Day 0. During Week-3, each mouse was uniquely identified by ear punch. On Day 0 of the study, the mice were approximately 16 weeks of age and weighed 20.0-25.0 kg.

Certified rodent diet #2016C (Harlan; Madison, Wis.) was supplied ad libitum during the pre-study and study periods. Tap water was provided ad libitum during the pre-study and study periods. The mice were group housed (maximum of 10/cage/sex/strain) in a room maintained at a temperature of approximately 68-74° F. and a relative humidity of approximately 48%-53%. Heat-treated hardwood chips were used in the bottom of the cages for bedding. No known contaminants were present in the food, water, or bedding that could interfere with or affect the outcome of the study. Room lights were controlled by an automatic timer set to provide 12 hours of light (0600 to 1800 hours, CST) and 12 hours of dark per day. Cage size and animal care conformed to the guidelines of the Guide for the Care and Use of Laboratory Animals, the U.S. Department of Agriculture through the Animal Welfare Act (Public Law 99-198), and to the applicable Standard Operating Procedures (SOPs) of Southern Research.

Test Article A (Amikacin), Test Article B (Levofloxacin), and Test Article C (Metronidazole) were received from Nostrum Technologies, LLC. The test articles were received at room temperature and stored at ≦25° C. and considered stable when so stored. Test Article D [Actimmune® (IFN-γ)], was received from Nostrum Technologies, LLC. The test article was received on ice packs and stored at 2-8° C. and considered stable when so stored.

The vehicle for Actimmune® (Dilution solution excipient) was received from Nostrum Technologies, LLC. The vehicle was received room temperature and stored at ≦25° C. and considered stable when so stored.

Actimmune® (IFN-γ; Interferon gamma-1b) was diluted in the dilution solution excipient as per manufacturer's directions. The contents of one (1) vial (100 μg/0.5 mL) were gently diluted to a final volume of 700 mL using the dilution solution excipient. This represented a 1:1400 dilution. Test articles A-C were supplied in a ready to use form and no additional formulations were required. All residual test articles were stored at room temperature (15-30° C.) or refrigerated (2-8° C.).

In order to obtain groups that were comparable by body weight, all mice were assigned to their respective treatment groups using a computer-generated randomization procedure. The body weights required for randomization were determined in Week-1. After randomization, mice were assigned to one of four groups as indicated below in Table 7.

TABLE 7

| | | | | Number of Mice | |
|---|---|---|---|---|---|
| | | Dose | Dose | Sub- | Sub- |
| Dose | | Level* | Volume | group | group |
| Group | Test Article | (mg/animal) | (μL/dose) | A | B |
| 1 | Amikacin | 0.480 | 100 | 3 | 3 |
| | (Test Article A) | | | | |
| 2 | Levofloxacin | 0.300 | 100 | 3 | 3 |
| | (Test Article B) | | | | |
| 3 | Metronidazole | 0.100 | 100 | 3 | 3 |
| | (Test Article C) | | | | |
| 4 | Actimmune | 0.00002 | 100 | 3 | 3 |
| | (Test Article D) | | | | |

*The actual delivered dose (reflects an approximate 50% loss on delivery due to inefficiencies in nebulization drug delivery) for the test articles were as follows (mg/animal): Amikacin 0.240, Levofloxacin 0.150, Metronidazole 0.05 and Actimmune 0.000014.

For microsprayer dosing via endotracheal administration, mice were anesthetized by Ketamine/Xylazine (K/X) sedation (50 mg/kg Ketamine and 5 mg/kg Xylazine) administered intraperitoneally (IP). In the event that the Ketamine/Xylazine sedation did not provide the desired level of anesthesia (animal was still active at time of challenge), Isoflurane was used via vaporizer to effect. A Bair Hugger warm air unit was used to keep animals warm during recovery from K/X anesthesia (i.e. after dosing).

On Day 0, mice were endotracheally intubated with a MicroSprayer® Aerosolizer (Penn-Century™, Inc.; Wyndmoor, Pa.) and test articles were delivered in the airways at a dose volume of 100 μL/dose at various dose levels.

All mice were observed at least twice daily throughout the prestudy and study periods for signs of moribundity and mortality. Detailed observations were recorded prior to dosing and prior to euthanasia.

Body weights for all animals were obtained during Week-1 (randomization) and then prior to dosing on Day 0.

Animals were used for collection of blood samples and the entire lung for plasma and drug level determinations. Each mouse was anesthetized with CO₂/O₂ and terminally bled via retro-orbital into tubes containing K₂EDTA. Upon collection each blood sample was mixed by gentle inversion, placed on ice, and subsequently centrifuged to separate plasma. Plasma samples were processed on the day they were collected, or were snap frozen using liquid nitrogen and stored frozen (at or below −70° C.) until analyzed.

Animals in Subgroup A were used for collection of blood samples for plasma drug level determinations at the 1 hour post dose time point following Day 0 dosing; samples were collected within ±3% of target time. Animals in Subgroup B were used for collection of blood samples for plasma drug level determinations at the 8 hour post dose time point following Day 0 dosing; samples were collected within ±3% of target time (Table 8).

TABLE 8

| | | | | | | | Dose | Nominal Dose | | |
|---|---|---|---|---|---|---|---|---|---|---|
| | | | | | | | Delivered | Level² (mg/animal) | | |
| | | | | | | | using Penn | [For Reference | | |
| | | | | Dilution | | Dose | Century ® | Only: does not | Number of Mice | |
| Dose | | Formulation | | sent for | | Volume | Device | reflect ~50% | Subgroup | Subgroup |
| Group | Test Article | Made | | study | | (μL/dose) | (mg/animal) | nebulization losses] | A | B |
| 1 | Amikacin | 240 | mg/mL | 2.4¹ | mg/mL | 100 | 0.240 | 0.480 | 3 | 3 |
| | (Test Article A) | | | | | | | | | |
| 2 | Levofloxacin | 150 | mg/mL | 1.5¹ | mg/mL | 100 | 0.15 | 0.300 | 3 | 3 |
| | (Test Article B) | | | | | | | | | |
| 3 | Metronidazole | 50 | mg/mL | 0.5¹ | mg/mL | 100 | 0.05 | 0.100 | 3 | 3 |
| | (Test Article C) | | | | | | | | | |
| 4 | Actimmune | 100 | mcg/0.5 mL | 0.00014² | mg/mL | 100 | 0.000014 | 0.00002 | 3 | 3 |
| | (Test Article D) | | | | | | | | | |

¹Amikacin, Levofloxacin and Metronidazole diluted 1:100
²The actual delivered dose (reflects an approximate 50% loss on delivery due to inefficiencies in netrulization drug delivery) for the test articles were as follows (mg/animal): Amikacin 0.240, Levofloxacin 0.150, Metronidazole 0.05 and Actimmune 0.000014.
3 Actimmune 100 mcg/0.5 mL vial diluted to 700 mL

Immediately following each subgroups blood collection time point, each animal was euthanized using CO₂ and the entire lung was collected for tissue drug level determinations (within 15 minutes after the blood sample), weighed, and snap-frozen. Lung samples were snap frozen using liquid nitrogen or dry ice and stored frozen (at or below −70° C.) until analyzed. Following collection of lungs, carcasses were discarded without further evaluation. Animals in Groups 1-3 had aerosol content (ng/mL), plasma content (ng/mL) and lung level (ng/g) assessed for parent using an appropriate LC/MS/MS method. Animals in Group 4 had lung tissue and plasma assayed using a commercially available ELISA method. Residual plasma and lung samples (including lung homogenate) were stored frozen at or below −70° C. until properly discarded.

Clinical Observations data are summarized in Table 9. All animals survived to their scheduled euthanasia time point on Day 0 (1 hour or 8 hours post dosing). All animals were normal (no clinical abnormalities) prior to dosing on Day 0 and prior to euthanasia 1 hour or 8 hours post dosing.

TABLE 9
Day numbers relative to Start Date

| | | | | 0 | 0 |
|---|---|---|---|---|---|
| | | | | 1 hr | 8 hr |
| Group | Sex | Clinical Sign | 0 | post dose | post dose |
| 1 | f | ANIMALS ALIVE | 6 | 6 | 3 |
| | | ANIMALS NORMAL | 6 | 3 | 3 |
| | | Scheduled euthanasia | — | 3 | 3 |
| 2 | f | ANIMALS ALIVE | 6 | 6 | 3 |
| | | ANIMAL NORMAL | 6 | 3 | 3 |
| | | Scheduled euthanasia | — | 3 | 3 |
| 3 | f | ANIMALS ALIVE | 6 | 6 | 3 |
| | | ANIMAL NORMAL | 6 | 3 | 3 |
| | | Scheduled euthanasia | — | 3 | 3 |
| 4 | f | ANIMALS ALIVE | 6 | 6 | 3 |
| | | ANIMALS NORMAL | 6 | 3 | 3 |
| | | Scheduled euthanasia | — | 3 | 3 |

Body weight data are summarized in Table 10. Mean body weights on Day 0 were 22.48 grams, 22.62 grams, 21.77 grams, and 22.72 grams for Groups 1-4, respectively. Individual body weights for each of the animals in Groups 1-4 ranged from 20.1 grams to 23.7 grams in Group 1, 20.1 grams, to 25.0 grams in Group 2, 20.0 grams to 24.4 grams in Group 3, and 20.9 grams to 24.6 grams in Group 4.

TABLE 10
Bodyweight (grams)
Day numbers relative to Start Date

| Group | Sex | | −20 | 0 |
|---|---|---|---|---|
| 1 | f | Mean | 20.82 | 22.48 |
| | | S.D. | 0.76 | 1.30 |
| | | N | 6 | 6 |
| 2 | f | Mean | 20.87 | 22.62 |
| | | S.D. | 0.86 | 1.56 |
| | | N | 6 | 6 |
| 3 | f | Mean | 20.80 | 21.77 |
| | | S.D. | 0.84 | 1.44 |
| | | N | 6 | 6 |
| 4 | f | Mean | 20.65 | 22.72 |
| | | S.D. | 0.90 | 1.56 |
| | | N | 6 | 6 |

Amikacin, Levofloxacin, and Metronidazole (Groups 1-3) levels evaluated in the plasma and lung tissues are presented in Table 11; and Actimmune levels (Group 4) evaluated in the plasma and lung tissues are presented in Table 12 and 13 respectively. The dosing solutions that were received and prepared by the sponsor were evaluated after the dosing was complete. The results indicated that the drug concentration levels for the solutions were 1.11 mg/mL to 1.62 mg/mL for Amikacin, 1.62 mg/mL to 1.65 mg/mL for Levofloxacin, and 0.462 mg/mL to 0.463 mg/mL for Metronidazole.

TABLE 11
Plasma and Lung Concentrations in Mice

| | Lung | | | | | | Lung |
|---|---|---|---|---|---|---|---|
| | Tissue | Nominal | | | Plasma | Lung | Concentration |
| | Weight | Timepoint | Route of | Dose | Concentration | Concentration | (μg/g) of |
| Animal ID | (mg) | (hr) | Administration | (mg/animal) | (ng/mL) | (ng/g) | Tissue |
| 1F 1 | 146.0 | 1 | IT | 0.48 | 4820 | 9480 | 64.9 |
| 1F 2 | 147.8 | 1 | IT | 0.48 | 2300 | 3990 | 27.0 |
| 1F 3 | 143.8 | 1 | IT | 0.48 | 7140 | 4830 | 33.6 |
| 1F 4 | 161.2 | 8 | IT | 0.48 | 406 | 3060 | 19.0 |
| 1F 5 | 145.1 | 8 | IT | 0.48 | 156 | 443 | 3.05 |
| 1F 6 | 176.2 | 8 | IT | 0.48 | 152 | 351 | 1.99 |
| G1 Dosing Solution | n/a | n/a | n/a | 2.4 mg/mL | 1.11 mg/mL | | |
| G1 Dosing Solution | n/a | n/a | n/a | 2.4 mg/mL | 1.33 mg/mL | | |
| G1 Dosing Solution | n/a | n/a | n/a | 2.4 mg/mL | 1.62 mg/mL | | |
| 2F 7 | 155.6 | 1 | IT | 0.3 | 556 | 2140 | 13.8 |
| 2F 8 | 134.2 | 1 | IT | 0.3 | 815 | 2270 | 16.9 |
| 2F 9 | 154.5 | 1 | IT | 0.3 | 534 | 1610 | 10.4 |
| 2F 10 | 165.4 | 8 | IT | 0.3 | BQL | BQL | n/a |
| 2F 11 | 149.5 | 8 | IT | 0.3 | BQL | BQL | n/a |
| 2F 12 | 158.8 | 8 | IT | 0.3 | 53.0 | 1200 | 7.56 |
| G2 Dosing Solution | n/a | n/a | n/a | 1.5 mg/mL | 1.64 mg/mL | | |
| G2 Dosing Solution | n/a | n/a | n/a | 1.5 mg/mL | 1.62 mg/mL | | |
| G2 Dosing Solution | n/a | n/a | n/a | 1.5 mg/mL | 1.65 mg/mL | | |
| 3F 13 | 140.6 | 1 | IT | 0.1 | 2290 | 1640 | 11.7 |
| 3F 14 | 164.5 | 1 | IT | 0.1 | 1670 | 1070 | 6.50 |
| 3F 15 | 153.5 | 1 | IT | 0.1 | 1880 | 1270 | 8.27 |
| 3F 16 | 161.5 | 8 | IT | 0.1 | 42.3 | BQL | n/a |
| 3F 17 | 160.8 | 8 | IT | 0.1 | BQL | BQL | n/a |
| 3F 18 | 153.1 | 8 | IT | 0.1 | BQL | BQL | n/a |

| | | | | | |
|---|---|---|---|---|---|
| G3 Dosing Solution | n/a | n/a | n/a | 0.5 mg/mL | .462 mg/mL |
| G3 Dosing Solution | n/a | n/a | n/a | 0.5 mg/mL | .463 mg/mL |
| G3 Dosing Solution | n/a | n/a | n/a | 0.5 mg/mL | .463 mg/mL |

BQL = below quantitation limit
IT = intratracheal dosing
Group 1 = 100-25,000 ng/mL (plasma) or 500-125,000 ng/g (hmg)
Group 2 & 3 = 25.0-10,000 ng/mL (plasma) or 125-50,000 ng/g (hmg)

TABLE 12
Human IFN-γ Levels in Mouse Lung Following Aerosol Delivery

| | | | Tissue | IFN-g | ng of |
|---|---|---|---|---|---|
| | | Animal | Weight | Level | IFN-g per |
| Group | Treatment | ID | (mg) | (pg/mL) | gm of tissue^{a} |
| 4 | Actimmune | 4F19 | 148.4 | 1732.7 | 116.8 |
| (Subgroup | (Test Article D) | 4F20 | 142.1 | 1248.4 | 87.9 |
| A) | 1 hr post dose | 4F21 | 147.2 | 1109.4 | 75.4 |
| | | Mean | | | 93.3 |
| | | SD | | | 21.2 |
| 4 | Actimmune | 4F22 | 172.6 | 1006.2 | 58.3 |
| (Subgroup | (Test Article D) | 4F23 | 218.6 | 1578.0 | 72.2 |
| B) | 8 hr post dose | 4F24 | 127.4 | 1307.7 | 102.6 |

| | |
|---|---|
| Mean | 77.7 |
| SD | 22.7 |

^{a}Tissues were homogenized in 10 mL of buffer containing protease inhibitors and assayed for human IFN-g levels by ELISA.

TABLE 13
IFN-γ Levels in Mouse Plasma Following Aerosol Delivery

| Group | Treatment | Animal ID | IFN-g Level (pg/mL) |
|---|---|---|---|
| 4 | Actimmune | 4F19 | BDL^{a} |
| (Subgroup A) | (Test Article D) | 4F20 | BDL |
| | 1 hr post dose | 4F21 | BDL |
| | | Mean | NA |
| | | SD | NA |
| 4 | Actimmune | 4F22 | BDL |
| (Subgroup B) | (Test Article D) | 4F23 | BDL |
| | 8 hr post dose | 4F24 | BDL |
| | | Mean | NA |
| | | SD | NA |

^{a}Below detectable levels (BDL). Mouse plasma was diluted 1:2 and assayed for IFN-g levels by ELISA with a reported sensitivity <2 pg/mL.

Plasma levels in Group 1, for the animals euthanized 1 hour post dosing (Subgroup A) with Amikacin, ranged from 2300 ng/mL to 7140 ng/mL and lung levels ranged from 3990 to 9480 ng/g of tissue. Plasma and lung levels were lower at the 8 hour time point (Subgroup B) and ranged from 152 ng/mL to 406 ng/mL for Amikacin levels in the plasma samples and 351 to 3060 ng/g of tissue for Amikacin levels in the lung samples.

Plasma levels in Group 2, for the animals euthanized 1 hour post dosing (Subgroup A) with Levofloxacin, ranged from 534 ng/mL to 815 ng/mL and lung levels ranged from 1610 to 2270 ng/g of tissue. Plasma and lung levels were lower at the 8 hour time point (Subgroup B) and were 53 ng/mL for Levofloxacin levels in the plasma and 1200 ng/g of tissue for Levofloxacin levels in the lung. Two of the three animals (2F10 and 2F11) evaluated in Group 2 at the 8 hour time point had plasma concentration levels and lung levels that could not be detected, therefore the reportable level of Levofloxacin in the plasma and lung samples was for a single animal (2F12) at the 8 hour time point.

Plasma levels in Group 3, for the animals euthanized 1 hour post dosing (Subgroup A) with Metronidazole, ranged from 1670 ng/mL to 2290 ng/mL and lung levels ranged from 1070 to 1640 ng/g of tissue. Plasma and lung levels were lower at the 8 hour time point (Subgroup B) and were 42.3 ng/mL in the plasma and not detectable for Metronidazole levels in the lung. Metronidazole levels in the lung were not detectable for all three animals at the 8 hour time point. Two of the three animals (3F17 and 3F18) evaluated in Group 3 at the 8 hour time point had plasma concentration levels that could not be detected, therefore the reportable level of Metronidazole in the plasma samples was for a single animal (3F16) at the 8 hour time point.

Plasma levels in Group 4 (administered Actimmune), for the animals euthanized 1 hour post dosing (Subgroup A) and 8 hours post dosing (Subgroup B), were not detectable. Lung levels for Actimmune 1 hour post dosing ranged from 75.4 to 116.8 ng of IFN-γ per gram of tissue and 58.3 to 102.6 ng of IFN-γ per gram of tissue 8 hours post dosing.

This study evaluated the plasma and lung levels of FDA approved antibiotic agents (Amikacin, Levofloxacin, and Metronidazole) and Actimmune® (IFN-γ; Interferon gamma-1b) in mice following Penn-Century™ MicroSprayer® aerosol (endotracheal) delivery. On Day 0, mice were endotracheally administered Amikacin (Group 1), Levofloxacin (Group 2), Metronidazole (Group 3) and Actimmune® (Group 4). Animals in each group were euthanized 1 hour post dose (3 animals per group) or 8 hours post dose (3 animals per group) in order to assess the plasma and lung levels.

Overall, the levels of Amikacin, Levofloxacin, Metronidazole, and Actimmune® (IFN-γ; Interferon gamma-1b) in the plasma and lungs decreased 8 hours post dose compared to the levels present 1 hour post dose. All animals in Group 1 administered Amikacin had detectable plasma and lung levels. Amikacin plasma levels 1 hour post dose ranged from 2300 ng/mL to 7140 ng/mL and 152 ng/mL to 406 ng/mL 8 hours post dose. Amikacin lung levels 1 hour post dose ranged from 3990 to 9480 ng/g of tissue and 351 to 3060 ng/g of tissue 8 hours post dose. The lung levels were higher compared to the plasma levels at both time points. The endotracheal administration of Amikacin via the Penn-Century™ MicroSprayer® appeared to result in detectable levels in the plasma and lungs 1 and 8 hours post dosing.

Levofloxacin (Group 2) was also detectable 1 hour post dose in the plasma and lung samples however, was only detectable in a single animal (1 of 3 animals) 8 hours post dose. This could indicate that the dosing site was missed for the two animals that had samples collected 8 hours post dosing. This is a possibility with the Penn-Century™ MicroSprayer® method and correct positioning of the microsprayer at the time of dosing was crucial. Also, the levels for all test articles were decreased 8 hours post dose so this may have been a combination of a missed dose and/or a partial dose with decreased levels at 8 hours post dose. It is hard to determine the exact reason for the undetectable levels. Levofloxacin plasma levels 1 hour post dose ranged from 534 ng/mL to 815 ng/mL and 53.0 ng/mL (for the single animal) 8 hours post dose. Levofloxacin lung levels 1 hour post dose ranged from 1610 to 2270 ng/g of tissue and 1200 ng/g of tissue (for the single animal) 8 hours post dose. Plasma and lung levels followed the same trend as the Amikacin levels in which lung levels were increased compared to the plasma levels. This same trend of increased lung levels compared to plasma levels was observed with the administration of metronidazole.

Metronidazole (Group 3) was detectable 1 hour post dose in the plasma and lung samples however, was only detectable for a single animal in the plasma 8 hours post dosing. These results also indicate that the dosing site for two animals may have been missed and/or a combination of a partial dose with decreased levels at 8 hours post dose. Again, the reason for the undetectable levels is hard to determine. Metronidazole plasma levels 1 hour post dose ranged from 1670 ng/mL to 2290 ng/mL and 42.3 ng/mL (for the single animal) 8 hours post dose. Metronidazole lung levels 1 hour post dose ranged from 1070 to 1640 ng/g of tissue and were not detectable 8 hours post dose.

Actimmune lung levels ranged from 75.4 to 116.8 ng of IFN-γ per gram of tissue 1 hour post dose and 58.3 to 102.6 ng of IFN-γ per gram of tissue 8 hours post dose. As with the Amikacin, Levofloxacin, and Metronidazole levels the Actimmune levels were also decreased 8 hours post dose. The Actimmune levels were below detectable levels (BDL) in the plasma samples 1 and 8 hours post dose.

These experiments, summarized in Table 14, demonstrate that the endotracheal administration of Amikacin, Levofloxacin, Metronidazole, and Actimmune® (IFN-γ; Interferon gamma-1b) via the Penn-Century™ MicroSprayer® was able to produce detectable levels in the lungs 1 hour post dose. Amikacin, Levofloxacin, and Metronidazole plasma levels were detectable 1 hours post dose however, Actimmune® (IFN-γ; Interferon gamma-1b) levels were not detectable in the plasma 1 or 8 hours post dose. Lung and plasma levels decreased 8 hours post dose compared to 1 hour post dose and not all test articles were detectable in the lung and plasma 8 hours post dose. Although, levels decreased 8 hours post dose, this model did demonstrate that the antitubercular agents can be delivered endotracheally and detected in the lungs and/or plasma 1 hour post dose.

TABLE 14

| | AMIKACIN | LEVOFLOXACIN | METRONIDAZOLE¹ | INTERFERON-gamma |
|---|---|---|---|---|
| Delivery Device (Intra-tracheal) | Penn Century ® | Penn Century ® | Penn Century ® | Penn Century ® |
| | MicroSprayer | MicroSprayer | MicroSprayer | MicroSprayer |
| Dose Delivered per Animal | 240 μg in 100 μL | 150 μg in 100 μL | 50 μg in 100 μL | 0.014 μg in 100 μL |
| Analytical Recovery Efficiency² | 56% | 109% | 93% | Not Applicable |
| Number of Animals/Time point | 3 | 3 | 3 | 3 |
| Reported MIC³ | 0.5 μg/mL (500 ng/mL) | 0.03 μg/mL (30 ng/mL) | 250 μg/mL in (250,000 ng/mL) | Not Applicable |
| Lung Levels: 1 hour (Range) | 27,000 ng/g to 94,900 ng/g | 10,400 ng/g to 16,900 ng/g | 6500 ng/g to 11,700 ng/g | 75.4 ng/g to 116.9 ng/g |
| | of lung tissue | of lung tissue | of lung tissue | of lung tissue |
| Lung Levels: 8 hour (Range) | 1990 ng/g to 19,000 ng/g | 7590 ng/g - | Not detected | 58.3 ng/g to 102.6 ng/g |
| | of lung tissue | Not Determined⁴ | | of lung tissue |
| Plasma Levels: 1 hour (Range) | 2300 ng/mL to 7140 ng/mL | 534 ng/mL to 815 ng/mL | 1670 ng/mL to 2290 ng/mL | Not Detectable |
| Plasma Levels: 8 hour (Range) | 152 ng/mL to 406 ng/mL | 53 ng/mL - | 42.3 ng/mL - | Not Detectable |
| | | Not Determined | Not Determined | |

¹Metronidazole is being investigated as an anaerotic antibiotic for TB. Its safety, efficacy, dosing and MIC have not been established for treatment of TB/MDR TB.
²Lung and Plasma data reported have not been corrected to account for analytical recovery efficiencies.
³MIC data are reported for reference only and do not imply levels required for anti-TB activity in the lung.
⁴We speculate that technician error in executing intra-tracheal delivery to the animals may have contributed to partial or no dose being delivered to certain experimental animals, resulting in no drug levels being detected. This artifact was noted in earlier experimentation as well. Future studies will use rebulizer delivery to circumvent this shortfall.

1. A method of treating a condition chosen from tuberculosis, mycobacterium avium complex, ventilator assisted pneumonia, comprising administering, by inhalation, to a patient in need thereof a pharmaceutically acceptable amount of an interferon and at least one other therapeutic agent selected from the group of fluoroquinolones, aminoglycosides and nitroimidazoles; wherein the composition may be administered in combination or sequentially.

2. The method of claim 1, wherein the therapeutic agents are administered sequentially.

3. The method of claim 1, wherein the interferon is interferon gamma.

4. The method of claim 1, wherein the fluoroquinolone is levofloxacin.

5. The method of claim 1, wherein the aminoglycoside is amikacin.

6. The method of claim 1, wherein the nitroimidazole is metonidazole.

7. The method of claim 1, comprising administering a composition that includes interferon gamma, levofloxacin, amikacin and metronidazole.

8. The method of claim 1, comprising administering a composition that includes an amount of amikacin in the formulation of about 600 to about 1250 mg per 3 or 5 mL nebule; an amount of levofloxacin in the formulation of about 300 to about 1250 mg per 3 or 5 mL nebule; an amount of metronidazole in the formulation of about 150 to about 250 mg per 3 or 5 mL nebule; an amount of interferon in the formulation of about 0.03 to about 165 mcg per 3 or 5 mL nebule.

9. The method of claim 1, wherein the tuberculosis is multi-drug resistant tuberculosis.

10. (canceled)

11. (canceled)

12. An inhalable pharmaceutical composition comprising an interferon and at least one other therapeutic agent selected from the group of fluoroquinolones, aminoglycosides and nitroimidazoles; wherein the composition has a pH of about 2 to about 8 and a tonicity of about 200 to about 800 mOsm.

13. The composition of claim 12, wherein the interferon is interferon gamma.

14. The composition of claim 12, wherein the fluoroquinolone is levofloxacin.

15. The composition of claim 12, wherein the aminoglycoside is amikacin.

16. The composition of claim 12, wherein the nitroimidazole is metonidazole.

17. The composition of claim 12, wherein the composition comprises interferon gamma, levofloxacin, amikacin and metronidazole.

18. The composition of claim 12, wherein the composition includes an amount of amikacin in the formulation of about 600 to about 1250 mg per 3 or 5 mL nebule; an amount of levofloxacin in the formulation of about 300 to about 1250 mg per 3 or 5 mL nebule; an amount of metronidazole in the formulation of about 150 to about 250 mg per 3 or 5 mL nebule; an amount of interferon in the formulation of about 0.03 to about 165 mcg per 3 or 5 mL nebule.

19. The composition of claim 18, wherein the pH of the composition ranges from 2 to 6.

20. The composition of claim 12, wherein the composition is enhanced by a surfactant.

21. The composition of claim 20, wherein the surfactant is selected from the group of sorbitan trioleate, soya lecithin, lecithin, oleic acid, magnesium stearate and sodium lauryl sulfate.

22. An inhalable pharmaceutical composition comprising at least one therapeutic agent selected from the group of fluoroquinolones, aminoglycosides and nitroimidazoles; wherein the composition has a pH of about 2 to about 8 and a tonicity of about 200 to about 800 mOsm.

23. The composition of claim 22, wherein the fluoroquinolone is levofloxacin, the aminoglycoside is amikacin and the nitroimidazole is metonidazole.
